(19) United States
(12) Patent Application Publication
(10) Pub. No.: US 2025/0288417 A1
(43) Pub. Date: Sep. 18, 2025

(54) DEVICE AND METHOD FOR CARDIAC VALVE REDUCTION

(71) Applicants: North Carolina State University, Raleigh, NC (US); Duke University, Durham, NC (US)

(72) Inventors: Muath Bishawi, Durham, NC (US); Ken Gall, Durham, NC (US); Joseph B. Tracy, Raleigh, NC (US); Matt Clary, Raleigh, NC (US); Sydney Cook, Raleigh, NC (US)

(21) Appl. No.: 19/078,178

(22) Filed: Mar. 12, 2025

Related U.S. Application Data
(60) Provisional application No. 63/564,025, filed on Mar. 12, 2024.

(51) Int. Cl. A61F 2/24

(52) U.S. Cl. CPC A61F 2/2445; A61F 2210/009; A61F 2220/0025

(57) ABSTRACT

Various examples are provided related to a cardiac valve repair device. In one example, the device includes an outer structure with outer blocks, and inner blocks. Each outer block can include an outer magnet pack disposed within in outer block void, at least one tension wire channel extending through the outer block between a first end and a second end and along the outer block void, a connection point on each of the first end and the second end, and attachment openings on each of a first side and a second side extending between the first end and the second end. The outer structure can also include tension wires extending through corresponding tension wire channels and connectors that can couple the outer blocks. Each inner block includes an inner magnet pack disposed within an inner block void and attachment openings that correspond to the attachment openings of the outer blocks.

### CROSS REFERENCE TO RELATED APPLICATIONS

This application claims priority to, and the benefit of, U.S. provisional application entitled “DEVICE AND METHOD FOR CARDIAC VALVE REDUCTION” having Ser. No. 63/564,025, filed Mar. 12, 2024, which is hereby incorporated by reference in its entirety.

### STATEMENT REGARDING FEDERALLY SPONSORED RESEARCH OR DEVELOPMENT

This invention was made with government support under CMMI1663416 and IIP2141188 awarded by the National Science Foundation. The government has certain rights in the invention.

### BACKGROUND

Percutaneous techniques mitigate risks associated with open-heart surgery and have shorter recovery times, making them accessible for a wider patient population, thus expanding the market by including patients who are not candidates for open-heart surgery. Moreover, because heart-lung bypass is not required as it is in open-heart procedures, percutaneous techniques enable surgeons to deploy and optimize the performance of devices on a beating heart. These statements have been validated with the introduction and later widespread use of Transfemoral Aortic Valve Replacement (TAVR). For the mitral valve however, the field remains at its infancy. Current percutaneous techniques for repair have limited performance and safety problems.

### SUMMARY

Aspects of the present disclosure are related to a cardiac valve repair device and method of using the cardiac valve repair device. In one aspect, among others, a device includes an outer structure comprising a plurality of outer blocks, each outer block comprising an outer magnet pack disposed within an outer block void; at least one tension wire channel extending through the outer block between a first end and a second end of the outer block and along the outer block void; a connection point on each of the first end and the second end of the outer block; and one or more attachment openings on each of a first side and a second side extending between the first end and the second end, the one or more attachment openings on opposite sides of the outer block void; one or more tension wires extending through corresponding tension wire channels of the plurality of outer blocks; and a plurality of connectors configured to couple to at least one outer block at the connection point on the first or second end; and a plurality of inner blocks, each inner block comprising: an inner magnet pack disposed within an inner block void; and one or more attachment openings that correspond to the one or more attachment openings of the outer blocks. In one or more aspects, the outer structure can be positioned in at least one of a linear alignment or a circular alignment. The circular alignment can be created by applying tension through at least one of the one or more tension wires. A diameter of the outer structure positioned in the circular alignment can be decreased based at least in part on tension applied by the one or more tension wires. In various aspects, the outer blocks can be connected to the inner blocks by an attachment mechanism via the one or more attachment openings of the outer blocks and the one or more attachment openings of the inner blocks. The attachment mechanism can be a suture, a bolt, a staple, or an anchor. In various aspects, the connectors can be springs. The outer magnet pack can be detachably attached to the outer block void of each outer block and the inner magnet pack is detachably attached to the inner block void of the inner blocks. The outer magnet pack can be permanently attached to the outer block void of each outer block and the inner magnet pack is permanently attached to the inner block void of each inner block. In various aspects, each outer magnet pack can be aligned so that a polarity of the outer magnet packs are oriented in a common direction. In some aspects, the inner magnet pack can have an inner polarity and the outer magnet pack has an outer polarity such that the inner polarity and the outer polarity align the inner blocks to the outer blocks. In one or more aspects, the outer magnet pack comprises one or more magnets and the inner magnet pack comprise one or more magnets.

In another aspect, a method comprises inserting an outer structure on to an outer portion of a valve, the outer structure comprising one or more tension wires extending through each of a plurality of outer blocks comprising outer magnet packs, the outer structure initially in a substantially linear alignment; applying tension in at least one of the one or more tension wires of the outer structure to change the outer structure to a circular alignment; inserting a plurality of inner blocks comprising inner magnet packs on to an inner portion of the valve, the plurality of inner blocks positioned so that each inner block aligns with a corresponding outer block of the outer structure using magnetic interactions between the inner magnet pack and outer magnet pack; and attaching the outer blocks to the inner blocks with an attachment mechanism that pierces through the valve and links corresponding attachment openings of the inner blocks and the outer blocks. In one or more aspects, the method can comprise removing the inner magnet packs from an inner block void of the inner blocks; and removing the outer magnet packs from an outer block void of the outer blocks. The method can further comprise applying tension in the one or more tension wires to decrease a diameter of the circular alignment of the outer structure. In various aspects, each outer magnet pack can be aligned so that a polarity of the outer magnet packs are oriented in a common direction. The inner magnet packs can have an inner polarity and the outer magnet packs can have an outer polarity such that the inner polarity and the outer polarity align the inner blocks to the outer blocks. Each of the outer magnet packs can comprise one or more magnets and each of the inner magnet packs can comprise one or more magnets. The outer blocks of the outer structure can be linked via springs connected to each outer block at a connection point. In some aspects, the one or more tension wires can run through one or more corresponding tension wire channels of each of the outer blocks, each tension wire channel extending through each of the outer blocks between a first end and a second end of the outer block and along an outer block void.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B depict on example of an outer structure of the device with varying amounts of tension and elements, in accordance with various embodiments of the present disclosure.

FIGS. 2A-2D illustrate examples of outer blocks and inner blocks, in accordance with various embodiments of the present disclosure.

FIGS. 3A-3C depict an example of a device on an elastic elliptical disk to mimic an adult mitral valve annulus, in accordance with various embodiments of the present disclosure.

FIG. 3D illustrates an example of an anchor, in accordance with various embodiments of the present disclosure.

FIGS. 4A-4C illustrate an example of the device with a magnet pack including multiple magnets, in accordance with various embodiments of the present disclosure.

FIGS. 5A-5D illustrate another example of outer blocks and magnet packs, in accordance with various embodiments of the present disclosure.

FIGS. 6A-6C depict another example of the device, in accordance with various embodiments of the present disclosure.

FIGS. 7A-7C depict the device with various degrees of tension applied, in accordance with various embodiments of the present disclosure.

FIGS. 8A-8G illustrate a method of using the device to reduce the diameter of the mitral annulus, in accordance with various embodiments of the present disclosure.

FIG. 9 illustrates a method of inserting the device, in accordance with various embodiments of the present disclosure.

### DETAILED DESCRIPTION

Disclosed herein are various examples related to a cardiac valve repair device. While the present disclosure is described with respect to cardiac valve repair and specifically to mitral valve annuloplasty, it is to be understood that the disclosed systems and methods could equally be used in other surgical applications. In some examples, the present disclosure can be used in heart repair to clip or hold leaflets, or to deform the shape of the annulus.

Heart disease represents a variety of common and dangerous health conditions that accumulate to have consistently been the leading cause of death in the United States. Heart disease generally involves a disruption in which blood cannot efficiently reach the heart or be pumped from the heart to other locations in the body. One of the most common types of heart disease in the world is mitral valve regurgitation. Mitral valve regurgitation occurs when the leaflet at the annulus of the mitral valve does not completely seal the valve, causing blood to leak backwards in the valve. This prevents a significant amount of blood from reaching vital parts of the rest of one's body and puts stress on the walls of the heart.

To treat severe mitral valve regurgitation, open heart surgery may be required, but for those that do not qualify for open heart surgery or cannot deal with the recovery process associated with it, percutaneous surgeries are available. For example, transcatheter mitral valve repair is a type of transcatheter procedure for treating regurgitation by directly altering the mitral anatomy to restore its intended functionality. Unlike with the aortic valve, the location and orientation of the mitral valve makes it incredibly difficult to gain access to with a catheter. Currently, the safest method is deploying the catheter through the femoral artery in the groin and entering the mitral valve by piercing the atrial septum, which requires multiple sharp turns to align with the valve itself, limiting the width of the device to a few millimeters across. The adverse event rate of devices, especially with annuloplasty systems, also has an issue. For example, several devices use screws to anchor to the mitral annulus which can cause damage to surrounding anatomy or cause significant bleeding, and screws can come loose over time.

The present disclosure is related to a steerable catheter-delivered annuloplasty ring that can use magnetic self-assembly to act as a temporary anchoring device on the mitral valve annulus, in some examples. For example, the device can comprise a self-assembling and mechanically adjustable device for mitral valve repair, which can be referred to as a “MagRing.” The device can be a circular shaped structure that is designed for manipulation with catheter-based tools. The flexible design of the device can allow for adjustable shapes, linear for delivery and circular for application to the valve. In various examples, the device can be configured to be placed at the annulus of a valve (e.g., mitral valve), anchored to the annulus, and then optionally reduced in diameter, thus effectively reducing the diameter of the annulus and treating mitral regurgitation. For example, the device can provide significant annular diameter reduction to treat mitral regurgitation by safely and quickly anchoring the device directly to the annular tissue. In various examples, the anchoring mechanism of the device can come from pinching the annular tissue with magnets.

In various examples, the device can comprise a top ring that can be a series of five rigid housing units (or outer blocks), connected by flexible springs, while the bottom pieces (or inner blocks) also contain magnets but are not connected to each other. In other implementations, the series can include other numbers of blocks (e.g., 3-10 blocks). The blocks can be 3D printed, molded or formed using other appropriate casting method. The rigid units (blocks) each can contain a small magnet for magnetic assembly in the heart. When connected, the rigid segments and the springs form a linear (or substantially linear) but flexible chain that can be steered by pulling one or more tension wires that run through the entire device. Therefore, the device can be deployed through a catheter with a small diameter but can be steered to take the shape of the mitral valve annulus once inside the left atrium.

For example, the device could be attached to a leaflet of the mitral valve and move it using a magnet, to allow for a percutaneous mitral valve to be better seated. The device can allow for suturing components together through a tissue wall. Additionally, the present disclosure can be configured to change shape, resulting in a diameter reduction in the device and therefore the respective valve.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

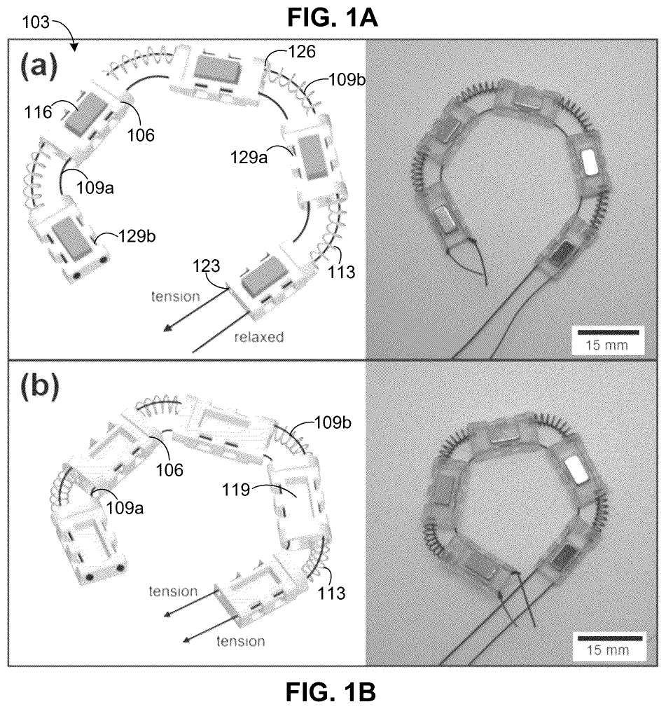

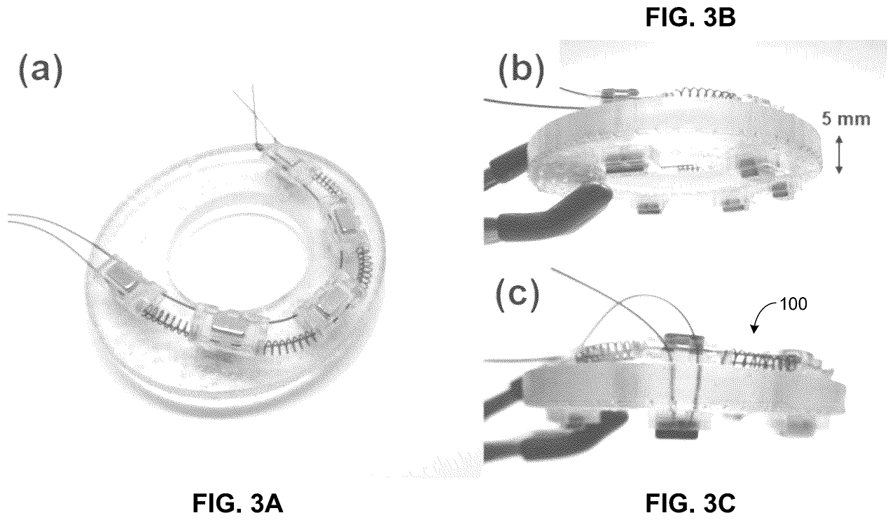

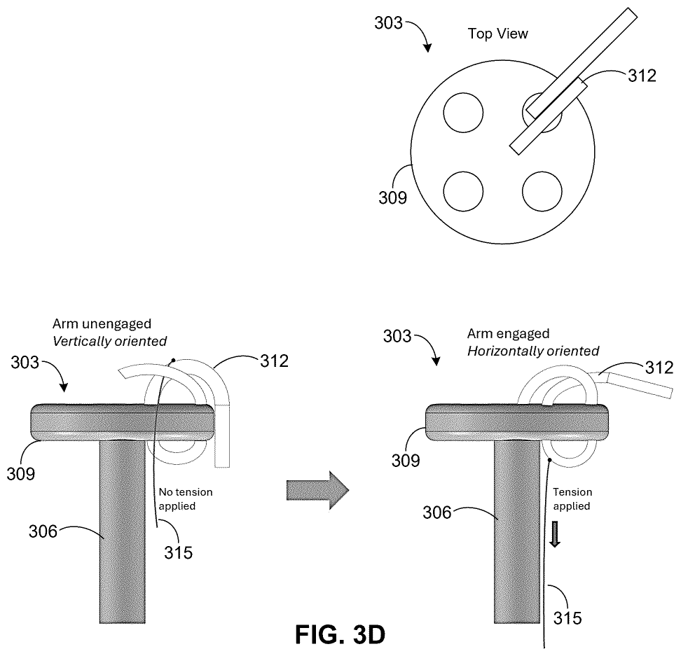

In various examples of the present disclosure, the device, or MagRing, 100 (FIG. 3C) of the present disclosure can comprise an outer structure 103 and a set of individual inner blocks 133. In reference to FIGS. 1A and 1B, shown is the top ring, or outer structure 103, of one example of the device 100. FIG. 1A shows the device 100 with magnets and FIG. 1B shows the device 100 with the magnet packs removed. According to various embodiments of the present disclosure, the outer structure 103 of the device 100 can include outer blocks 106, tension wires 109 (109a, 109b), and connectors (e.g., springs) 113.

The outer structure 103 includes multiple outer blocks 106 (five outer blocks 106 in the example of FIGS. 1A and 1B). Each individual outer block 106 can include a magnet pack 116, an outer block void 119, one or more tension wire channels 123, connection points 126, and attachment openings 129 (129a, 129b). The outer blocks 106 can be made of Formlabs BioMed Clear resin or other suitable biocompatible polymer, metal, ceramic, or composite with similar mechanical properties. The outer blocks 106 can have, e.g., a length in a range from about 15 mm to about 4 mm, a width from about 10 mm to about 2 mm, and a thickness from about 5 mm to about 0.5 mm. The outer block void 119 can be, e.g., about 15 mm to about 4 mm. In one example, the outer block 106 is 10.54 mm in length, 6.5 mm in width, and 2.5 mm in thickness with an outer block void 119 of 8.54 mm.

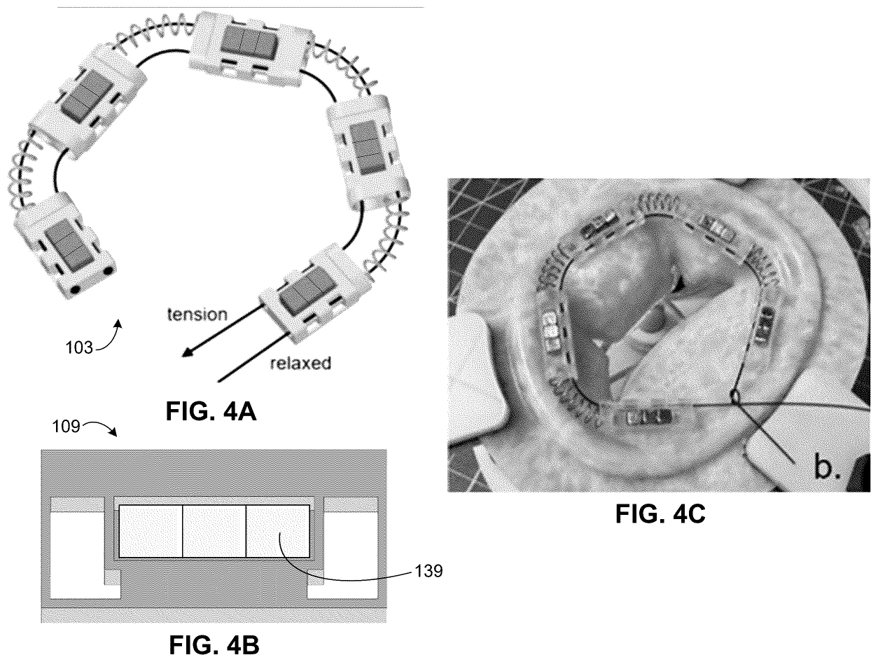

The magnet pack 116 of each outer block 106 can be permanently attached or detachably attached to the outer block 106 via the outer block void 119. In some examples, the magnet packs 116 can be aligned so that the polarity of the magnets are oriented in a common direction. The magnet packs 116 can be appropriately constructed to sit within the outer block void 119 of the outer blocks 106. The rectangular cross section with a length significantly longer than the width of the outer block void 119 can serve two purposes: to maximize the magnetic forces while maintaining a width and thickness small enough to fit through a catheter, and to increase the anisotropy of the magnetic field lines to improve alignment. In various examples of the present disclosure, one or more magnets (e.g., 1-10 cubes with edge lengths of, e.g., 1-6 mm) can be used in the magnet packs 116. For example, FIGS. 1A and 1B depict a singular magnet as the magnet pack 116. Alternatively, FIG. 4A illustrated the device 100 with sets of three magnets per magnet pack 116. Further, in some examples the magnet pack 116 can include an array of NdFeB permanent magnets for example, which are strong, widely available, and inexpensive. In some embodiments and as shown in FIGS. 4A-4C, the magnet pack 116 can contain pairs of cubic magnets (e.g., in a range from 2.25 mm to 4 mm edge length, or smaller), which generate strong forces. By adjusting the orientations of each cubic magnet and their relative orientation in the outer structure 103 and inner blocks 133, unique and tailored interaction strengths can be achieved and tuned from very strong to intermediate strength to weak attractions and repulsions. The magnet packs 116 will interact with each other through the non-magnetic material of the outer structure 103 and inner blocks 133, and magnetic assembly can also correct for a lateral offset in the alignment.

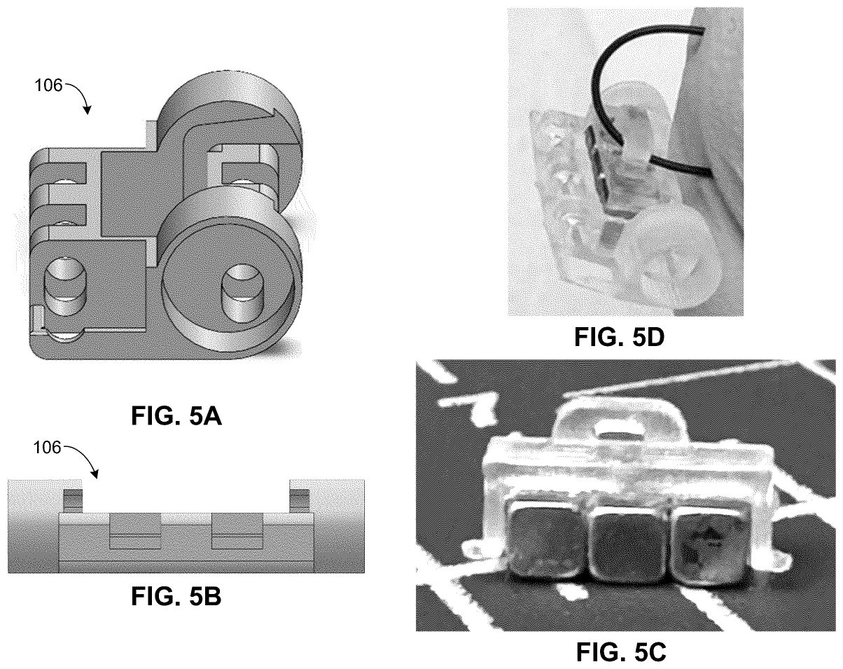

An example of a magnet pack 116 is illustrated in FIG. 4B and FIG. 5C. In these illustrations, the magnet pack 116 includes a structure employed to house the magnets within the blocks. For example, the structure shown in FIG. 5C allows for the magnet pack 116 to remove all of the magnets in one structure from the blocks. The use of the magnets in the device 100 can allow for imprecise movements while still achieving a connection and, if placed in the incorrect area, can be pulled apart and reconnected in the intended location. Additionally, magnetic pinching does not pierce or damage the surrounding tissue, allowing users to ensure the device is in the ideal location before progressing to more permanent fixation methods. In examples with one magnet per magnet pack 116, each magnet can be inserted into a centered rectangular hole (the outer block void 119) in the top ring's rigid housing unit. To prevent unwanted magnetic interactions between neighboring magnets in the top ring, the polarities of the magnets can be oriented in a common direction when inserted into the top ring. Due to the direction of the magnetic field lines stemming from each magnet, this orientation would cause repulsion of adjacent magnets if the ring were to bend or fold intentionally or unintentionally and prevent the pieces on the top ring from snapping together. In various examples, magnets could have a similar arrangement in the inner blocks 133.

Once again in reference to FIGS. 1A and 1B, the outer blocks 106 can also have one or more tension wire channels 123. The tension wire channels 123 can extend along the outer block void and through the outer block 106 from one end of the outer block 106 to the other. At the same ends of the outer block 106 comprising the beginning and end of the tension wire channels 123, the outer block 106 can have connection points. The sides of the outer blocks 106 can have attachment openings 129. In other words, the sides of the outer blocks 106 extending between the two ends contain one or more attachment openings 129 on opposite sides of the outer block void 119. In some examples, the attachment openings 129 can provide an opening for an attachment mechanism such as sutures, staples, etc.

Further in reference to FIGS. 1A and 1B, the tension wires 109 can be two wires as shown in FIGS. 1A and 1B. The tension wires 109 can run through corresponding tension wire channels 123 of the outer blocks 106, connecting the multiple outer blocks 106 together. The tension wires 109 can be made of, e.g., a high strength polymer, metal, ceramic, or composite to prevent deformation during the procedure while being unresponsive to the neighboring permanent magnets. Other non-magnetic medical grade materials (e.g., nylon suture thread) may also be utilized. The tension wires 109 can have a length in, e.g., a range from about 400 mm to about 12 mm. The tension wires 109 can be used to apply varying amounts of tension to different sides of the outer structure 103. For example, by applying tension through one of the tension wires 109, the device 100 could be arranged in a circular alignment forming an open ring. Further, tension can also be applied in the one or more tension wires 109 to decrease the diameter of the outer structure 103 when the outer structure 103 is positioned in the circular alignment and attached to the valve.

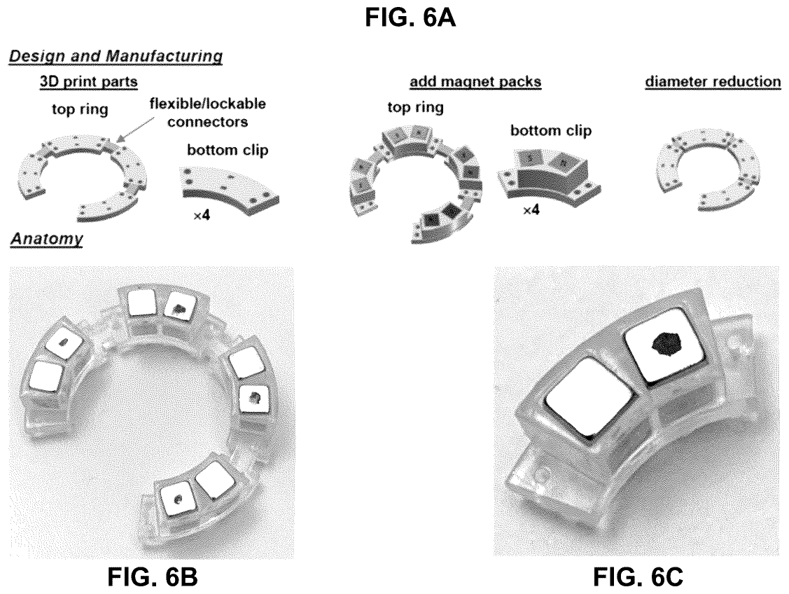

The connectors 113 can connect to each outer block 106 at the connection points 126. In various examples, the connectors 113 can be springs as shown in FIGS. 1A and 1B. In other examples, the connectors 113 can be composed of the same material as the outer blocks 103 as shown in FIGS. 6A-6C or any appropriately flexible substance or material. For example, the connectors can be stainless 302 ASTM A313 material type or other semi-rigid material with similar mechanical properties. The connectors can have a length in, e.g., a range from about 25 mm to about 2 mm. The connectors 113 promotes the flexible nature of the device 100 and allows for alignment changes where the outer structure 103 can be positioned in a circular alignment or a linear (or substantially linear) alignment. In other words, the connectors 113 in the device 100 provide mechanical compliance and allow for controlled manipulation that allow for a transcatheter annuloplasty procedure. In various examples, the connectors 113 can in part determine the size and shape of the device 100 when surrounding the annulus. In the example of a spring as a connector 133, the spring constants of the springs affect the amount of force needed to steer the device 100 and eventually reduce the device's 100 final diameter. The amount of force needed for diameter reduction can be calculated with a variation of Hooke's law, given by:

$$\vec{{F}_{T}} = \vec{{F}_{R}} + \vec{{F}_{L}} = {\sum }_{n = 1}^{4} - ( {k}_{n} * {\vec{x}}_{n} + {c}_{n} * {θ}_{n} )$$

where \vec{F_{T}} is the total force applied to both right (\vec{F_{R}}) and left (\vec{F_{L}}) tension wires, n represents a certain spring in the series with n=1 being the first spring to enter the catheter, k and c are the spring constant and torque constant of the spring, respectively, x is the displacement along the center axis of the spring, and θ is the angle of bending on the spring when the device is steered.

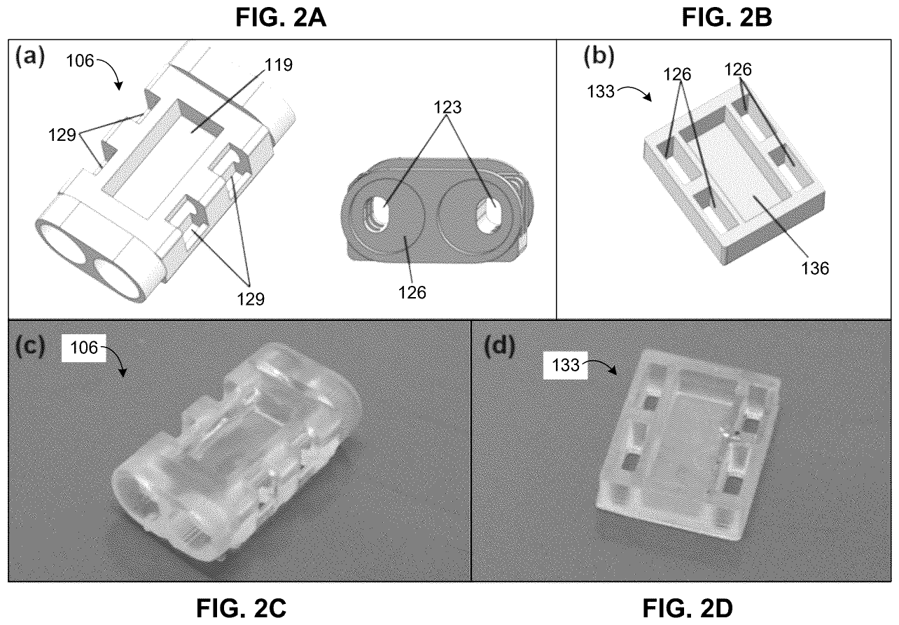

With reference to FIGS. 2A-2D, shown are various depictions of the outer blocks 106 and inner blocks 133. In reference to FIGS. 2A and 2C, depicted are illustrations of an example of an outer block 106 as described above having, an outer block void 119, tension wire channels 123, connection points 126, and attachment openings 129. FIGS. 2B and 2D illustrate one example of an inner block 133.

The inner blocks 133 can have a void 136. The void 136 of the inner blocks 133 can receive a magnet pack 116. Similarly to the outer blocks 106, the inner block 133 also has attachment openings 129. The attachment openings 129 of the inner blocks 133 can correspond to the attachment openings of the outer blocks 106. In some examples, the attachment openings 129 can provide an opening for attachment mechanisms to link the outer blocks 106 of the outer structure 103 to the inner blocks 133 through the tissue of the valve. The inner blocks 133 can be made of Formlabs BioMed Clear resin or other suitable biocompatible polymer, metal, ceramic, or composite with similar mechanical properties. The inner blocks 133 can have, e.g., a length in a range from about 15 mm to about 4 mm, a width from about 10 mm to about 2 mm, and a thickness from about 5 mm to about 0.5 mm. The inner block void 136 can be, e.g., about 15 mm to about 4 mm. In one example, inner blocks 133 is 10.54 mm in length, 6.5 mm in width, and 2.5 mm in thickness with an inner block void 136 of 8.54 mm.

Shown in FIGS. 3A-3C, use of the device 100 is illustrated by placing it on either side of a 5 mm thick elastic elliptical disk with comparable dimensions to an adult mitral valve annulus. In FIG. 3B, the inner blocks 133 are shown aligned with the outer blocks 106 via the magnetic attraction. For example, the magnet packs 116 of the outer blocks 103 can have an outer polarity and the inner blocks 133 can have an inner polarity such that the outer polarity and the inner polarity align each inner block 133 to each outer block 106. In other words, completion of magnetic assembly in the mitral valve relies on the secondary set of magnets to be placed on the sub-annular region of the valve to connect with the top ring magnets. Like the outer structure 103, magnets of the same dimensions and magnetization direction are inserted into rigid housing pieces (inner blocks 133) with a centered rectangular hole (inner block void 136). The polarity of the magnets in the inner blocks 133 are opposite to the polarity of those in the outer blocks 106, resulting in attractive forces between the top and bottom magnets when placed in proximity to each other. Alignment of each outer block 106 to its corresponding lower block 133 is important because the attachment openings 129 must align to ensure permanent anchoring of the device 100 to the annular tissue.

Physical attachment of the outer blocks 106 to the inner blocks 133 is shown in FIG. 3C. For example, the outer blocks 106 can be connected to the inner blocks 133 by an attachment mechanism via the one or more attachment openings 129 of the outer blocks 106 and the corresponding one or more attachment openings 129 of the inner blocks 133. In FIG. 3C, sutures are used to attach the inner blocks 133 to the outer blocks 106 through the disk.

While magnetic assembly can grip the annular tissue for placement, the holding force for the magnets may not be strong enough to hold the annulus without slipping during the diameter reduction stage. Therefore, a secondary, and more permanent, fixation method can be employed to anchor the device 100 to the annulus. For example, when magnetic assembly occurs between the outer blocks 106 and the inner blocks 133, due to the anisotropic shape of the permanent magnets, the attachment openings 129 can align (or nearly align) so that the attachment mechanism can be driven through the holes and the annular tissue. In some examples, the device 100 can attach itself to the attachment openings 129 of the top or bottom ring and drive a suture, a bolt or staple with the press of a button.

The attachment mechanism can also comprise anchors that extend through attachment openings 129 to sandwich the tissue of the annulus between the outer blocks 106 and the inner blocks 133. FIG. 3D illustrates an example of an anchor 303 comprising a cylinder or shaft 306 extending from a side of a circular disk 309, which includes a plurality of openings (e.g., 2, 3, 4, or more) surrounding the cylinder or shaft 306. The circular disk 309 can comprise a curved edge with the openings being distributed equidistant from the center and each other. The cylinder or shaft offers ease of handling and directional stability. The anchor 303 further includes a plurality of arms 312 (e.g., 2, 3, 4, or more) secured to openings of the circular disk 309. Each arm 312 is looped through a corresponding opening with a proximal end of the arm adjacent to the center of the circular disk 309 opposite the cylinder or shaft 306 and a distal end of the arm located outside of a circumference of the circular disk 309. In an unengaged position, the proximal end of the arm is not engaged with the circular disk 309 and a section of the arm extending from the loop through the corresponding opening to the distal end of the arm can extend substantially parallel to the cylinder or shaft 306 of the anchor 303. In an engaged position, the proximal end of the arm is engaged with the circular disk 309 opposite the cylinder or shaft 306 and the section of the arm extending from the loop to the distal end of the arm extends outward from the circular disk 309. Wires or sutures 315 (e.g., suture thread or other nylon based wire) can be attached to each arm 312 as shown in FIG. 3D. The wires or sutures 31 extend through the openings in the unengaged position and are pulled through the openings when in the engaged position. The circular disk 309 and cylinder or shaft 306 can be made of Formlabs BioMed Clear resin or other suitable biocompatible polymer, metal, ceramic, or composite with the similar mechanical properties. The arms 312 can be stainless steel or other suitable biocompatible metal or rigid plastic exhibiting suitable strength properties. The circular disk can be, e.g., about 2 mm in diameter with the openings having a inner hole diameter of, e.g., about 0.66 mm, and the cylinder length can be, e.g., about 2 mm. The dimensions can be varied depending on the application.

The device 100 can be anchored by sandwiching tissue of the annulus using the anchors 303 comprising multiple arms 312 that can be extended by pulling on the wires 315 attached to the arms 312. Without applying tension to the wires 315, the arms 315 are in the unengaged position, allowing for the anchor 303 to fit through the attachment openings 129. In the unengaged position, the anchor 303, with multiple arms 312 and no tension applied to the wires 315, is pushed past the attachment openings 129 of both the inner block 133 the outer block 106. The anchor 303 can be inserted with a needle with the circular disk 306 inserted through the attachment opening 129 first. Once the arms 312 have crossed the threshold of the attachment opening 129, tension can be applied to the wires 315, causing them to move to the engaged position, with the proximal end of each arm 312 preventing the arm from being pulled all the way to an outwardly extended position. The arms 312 thus blocks the attachment opening 129 and prevents the anchor 303 from passing back through the attachment opening 129. The needle can be removed and the wires can be tied off or a locking device applied to maintain tension and hold the anchors in place.

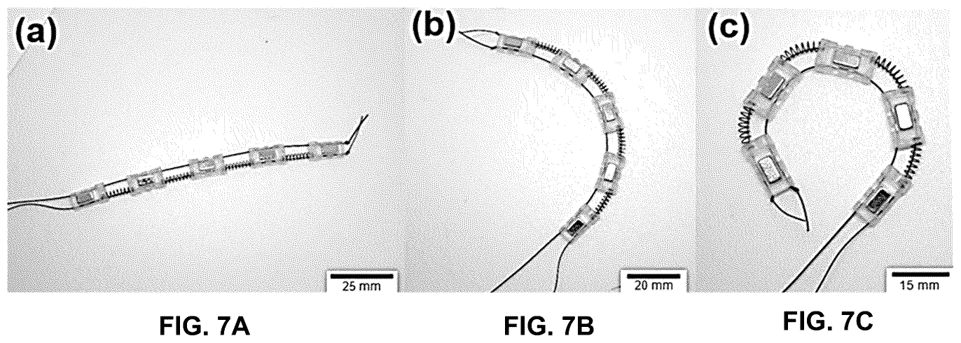

FIGS. 7A-7C show various arrangements of the device 100. In FIG. 7A, outer structure 103 is shown in a linear alignment. In FIG. 7B, the outer structure 103 is shown in a semi-circle arrangement and in FIG. 7C, the outer structure 103 is depicted in a circular arrangement. One function of the device 100 is transitioning from a linear series of components to a ring that encompasses the mitral valve as it enters the left atrium. The linear (or substantially linear) alignment allows the device 100 to be deliverable through a catheter small enough (e.g., less than ˜8 mm diameter) to travel through the patient's femoral artery. To enable this shape transition, a pair of tension wires 109 can be strung through the outer blocks 106 on both sides of the inserted magnets packs 116. To steer the device in each direction, one can apply tension to the corresponding tension wire 109, e.g. pulling the left wire will cause the device to bend to the left and vice versa. The directional pull from one side is exemplified in FIG. 7B. Applying a pulling force to a given side of the device can cause the connectors 113 in the system to compress and bend in that direction until it takes the shape of a ring.

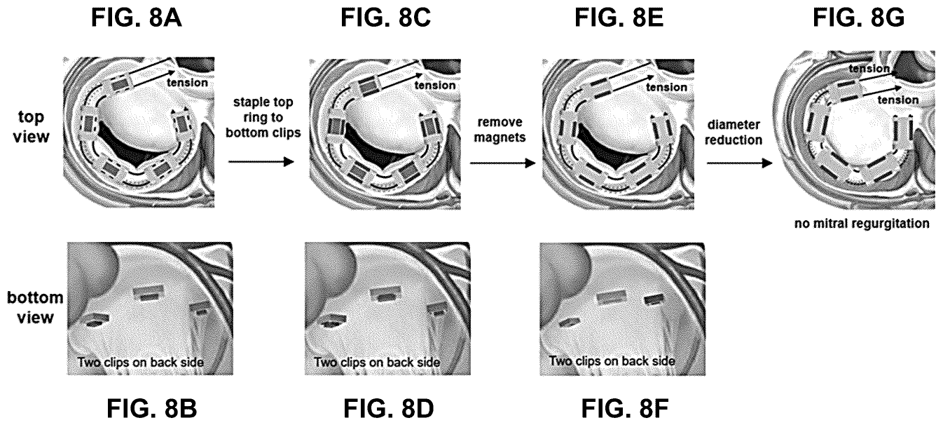

With reference to FIG. 8G, shown is the device 100 according to various embodiments of the present disclosure with diameter reduction. The tension wires 109 can serve as the driving force for reducing the diameter of the mitral annulus. Once the device 100 is permanently secured to the annular tissue via an attachment mechanism such as sutures, both the left and right tension wires 109 can be pulled to compress the connectors 113 while maintaining the curved configuration. This diameter reduction is also illustrated in FIGS. 1A and 1B. Following the diameter reduction process, the tension wires 109 can be fixed in place to hold the device 100 in the compressed state.

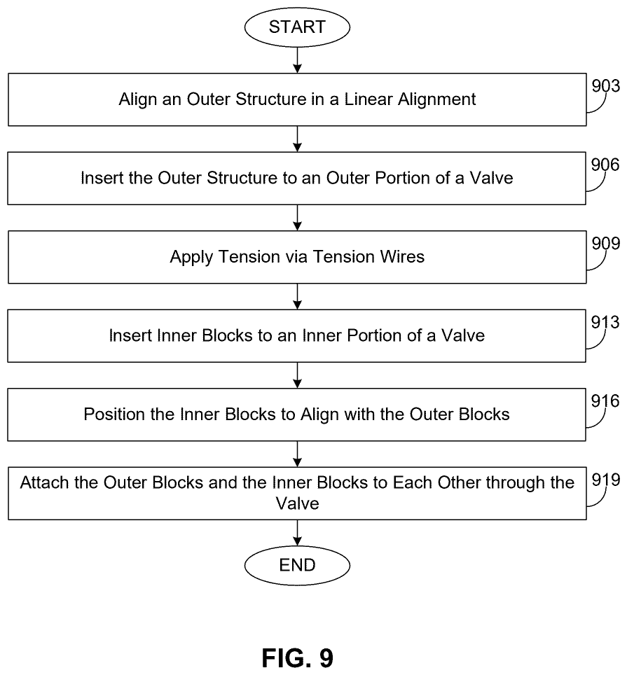

Referring next to FIG. 9, shown is a flowchart that provides one example of the operation of the device 100. The flowchart of FIG. 9 is illustrated further in FIGS. 8A-8G. The flowchart of FIG. 9 provides merely an example of the many different methods of application of the device 100 according to various embodiments of the present disclosure.

Beginning with block 903, the outer structure 103 of the device 100 can be aligned in a linear alignment. A linear (or substantially linear) alignment of the outer structure 103 can allow for the outer structure 103 to be insert into the body via a catheter. For example, the device 100 is a steerable catheter-deliverable annuloplasty ring and the linear alignment allows for catheter delivery.

Next, at block 906, the outer structure 103 of the device 100 can be inserted on to an outer portion of a valve. In various examples, delivery can occur via a catheter. Further, in some examples, the valve can be a mitral valve. During insertion of the outer structure 103, tension can be applied via the tension wires 109 to direct the linear outer structure 103 in a desired direction. For example, applying tension to the left tension wire 109 can steer the outer structure 103 in a left direction. Once a desired location is approached, the outer structure 103 can be placed on the outer portion of the valve.

At block 909, tension can be applied to one or more tension wires 109 of the outer structure 103. By applying tension to one of the tension wires 109, the outer structure 103 can change from a linear alignment to a circular alignment. The circular alignment allows for the outer structure 103 to form a shape that at least partially encloses the valve.

Next, at block 913, the inner blocks 133 can be inserted on to an inner portion of the valve. Similar to the outer structure 103, the inner blocks 133 can be inserted via catheter. For example, the inner blocks 133 can be deployed to the underside of the mitral valve.

Then at block 916, the inner blocks 133 can be positioned to align with the outer blocks 106. For example, the inner blocks 133 can be positioned so that the inner blocks 133 align with the outer blocks 106 of the outer structure 103 using magnetic interactions between the magnet packs 116 of the inner blocks 133 and magnet packs 116 of the outer blocks 106. In some examples, the alignment of the magnet packs 116 in this step, can further help align the inner blocks 133 to the outer blocks 106. This helps ensure that the respective attachment openings 129 of the outer blocks 106 and the inner blocks 133 coordinate with each other.

Finally, at block 919, the inner blocks 133 can be attached to the outer blocks 106 through the valve. For example, the inner blocks 133 can be attached to the outer blocks 106 via sutures or staples that pierce through the valve linking the attachment openings 129 of the outer blocks 106 to the attachment openings 129 of the inner blocks 133. The attachment mechanism can physically secure the outer structure 103 and the inner blocks 133 to the valve which can allow in some examples for reduction of the diameter of the device 100 and therefore a reduction in the diameter of the mitral valve.

The flow chart of FIG. 9 shows the method of a possible implementation of the device 100 as described above. Further, the method as described in the flowchart of FIG. 9 is illustrated in FIGS. 8A-8G. Although the flowchart shows a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowchart can be skipped or omitted.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. The term “about” can include traditional rounding according to significant figures of numerical values. In addition, the phrase “about ‘x’ to ‘y’” includes “about ‘x’ to about ‘y’”. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human who is undergoing a procedure using a system or method as prescribed herein.

Unless otherwise defined, all technical terms used herein have the same meaning as commonly understood by one of ordinary skill in the art to which this disclosure belongs.

Therefore, at least the following is claimed:

1. A device, comprising:
an outer structure, comprising:
a plurality of outer blocks, each outer block comprising:
an outer magnet pack disposed within an outer block void;
at least one tension wire channel extending through the outer block between a first end and a second end of the outer block and along the outer block void;
a connection point on each of the first end and the second end of the outer block; and
one or more attachment openings on each of a first side and a second side extending between the first end and the second end, the one or more attachment openings on opposite sides of the outer block void;
one or more tension wires extending through corresponding tension wire channels of the plurality of outer blocks; and
a plurality of connectors configured to couple to at least one outer block at the connection point on the first or second end; and
a plurality of inner blocks, each inner block comprising:
an inner magnet pack disposed within an inner block void; and
one or more attachment openings that correspond to the one or more attachment openings of the outer blocks.

2. The device of claim 1, wherein the outer structure is positioned in at least one of a linear alignment or a circular alignment.

3. The device of claim 2, the circular alignment is created by applying tension through at least one of the one or more tension wires.

4. The device of claim 2, wherein a diameter of the outer structure positioned in the circular alignment is decreased based at least in part on tension applied by the one or more tension wires.

5. The device of claim 1, wherein the outer blocks are connected to the inner blocks by an attachment mechanism via the one or more attachment openings of the outer blocks and the one or more attachment openings of the inner blocks.

6. The device of claim 5, wherein the attachment mechanism is a suture, a bolt, a staple, or an anchor.

7. The device of claim 1, wherein the connectors are springs.

8. The device of claim 1, wherein the outer magnet pack is detachably attached to the outer block void of each outer block and the inner magnet pack is detachably attached to the inner block void of the inner blocks.

9. The device of claim 1, wherein the outer magnet pack is permanently attached to the outer block void of each outer block and the inner magnet pack is permanently attached to the inner block void of each inner block.

10. The device of claim 1, wherein each outer magnet pack is aligned so that a polarity of the outer magnet packs are oriented in a common direction.

11. The device of claim 1, wherein the inner magnet pack has an inner polarity and the outer magnet pack has an outer polarity such that the inner polarity and the outer polarity align the inner blocks to the outer blocks.

12. The device of claim 1, wherein the outer magnet pack comprises one or more magnets and the inner magnet pack comprise one or more magnets.

13. A method, comprising:
inserting an outer structure on to an outer portion of a valve, the outer structure comprising one or more tension wires extending through each of a plurality of outer blocks comprising outer magnet packs, the outer structure initially in a substantially linear alignment;
applying tension in at least one of the one or more tension wires of the outer structure to change the outer structure to a circular alignment;
inserting a plurality of inner blocks comprising inner magnet packs on to an inner portion of the valve, the plurality of inner blocks positioned so that each inner block aligns with a corresponding outer block of the outer structure using magnetic interactions between the inner magnet pack and outer magnet pack; and
attaching the outer blocks to the inner blocks with an attachment mechanism that pierces through the valve and links corresponding attachment openings of the inner blocks and the outer blocks.

14. The method of claim 13, further comprising:
removing the inner magnet packs from an inner block void of the inner blocks; and
removing the outer magnet packs from an outer block void of the outer blocks.

15. The method of claim 13, further comprising applying tension in the one or more tension wires to decrease a diameter of the circular alignment of the outer structure.

16. The method of claim 13, wherein each outer magnet pack is aligned so that a polarity of the outer magnet packs are oriented in a common direction.

17. The method of claim 13, wherein the inner magnet packs have an inner polarity and the outer magnet packs have an outer polarity such that the inner polarity and the outer polarity align the inner blocks to the outer blocks.

18. The method of claim 13, wherein each of the outer magnet packs comprise one or more magnets and each of the inner magnet packs comprise one or more magnets.

19. The method of claim 13, wherein the outer blocks of the outer structure are linked via springs connected to each outer block at a connection point.

20. The method of claim 13, wherein the one or more tension wires run through one or more corresponding tension wire channels of each of the outer blocks, each tension wire channel extending through each of the outer blocks between a first end and a second end of the outer block and along an outer block void.
